(19) United States
(12) Patent Application Publication
(10) Pub. No.: US 2005/0196371 A1
(43) Pub. Date: Sep. 8, 2005

(54) Aqueous composition based on alcohol, surfactant and solubilizing agent, article impregnated with this composition, and method for cleansing the hair

(75) Inventors: Sandrine Decoster, Saint Gratien (FR); Virginie Masse, Clichy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/074,201

(22) Filed: Mar. 8, 2005

Related U.S. Application Data
(60) Provisional application No. 60/550,736, filed on Mar. 8, 2004.

(30) Foreign Application Priority Data
Mar. 8, 2004 (FR) 0450465

(51) Int. Cl. A61K 7/06; A61K 7/075; A61K 7/08

(52) U.S. Cl. 424/70.21

(57) ABSTRACT

The present invention relates to a hair composition preferably intended for cleansing the hair, containing, in a cosmetically acceptable aqueous medium, at least one C2-C4 monoalcohol, at least one surfactant and at least one agent that solubilizes liposoluble compounds, and to a method for cleansing said hair using this composition. It also relates to a cosmetic article impregnated with this composition and to the use of this composition for cleansing the hair, and to a method for cleansing said hair using this composition or this article.

### REFERENCE TO PRIOR APPLICATIONS

This application claims priority to U.S. provisional application 60/550,736 filed Mar. 8, 2004, and to French patent application 0450465 filed Mar. 8, 2004, both incorporated herein by reference.

### FIELD OF THE INVENTION

The present invention relates to a composition preferably intended for cleansing the hair, comprising water, at least one C2-C4 monoalcohol, at least one surfactant, and at least one agent that solubilizes liposoluble compounds, and to methods for cleansing wet and dry hair using this composition. Articles impregnated with this composition, and methods using the invention articles, also make up a part of the invention.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The hair has a tendency to lose some of its qualities under the action of factors such as natural greasing, sweat, the elimination of squamae, pollution, humidity, and the like. These factors are harmful to the visual appearance and to the feel of the hair. Thus, regreasing (and possibly pollution) weigh down the hair, which has a tendency to clump together. The hair is then difficult to style, it has an unpleasant greasy sheen and is waxy to the touch, which is also unpleasant.

The extent of the consequences of these factors, which are almost all inevitable, is very variable. It depends, for example, on the quality of the hair, on its length, and on the hairstyle adopted.

Be that as it may, shampoos are used to combat these inconveniences. In fact, washing with detergent compositions is very effective; it makes it possible to remove dirt and dandruff. It is then possible, during drying, to return the hair to the desired shape. However, the beneficial effect of the shampoo fades and, in a few days, the problems described above return. Consequently, there is a tendency to increase the frequency of shampooing.

To shampoo the hair, it is necessary to have a source of water, preferably hot or warn water.

Shampoo compositions are based on large amounts of surfactants which can generate inconveniences such as stinging of the scalp or of the eyes.

The use of “dry” shampoos has already been proposed in order to cleanse the hair more rapidly and to avoid wetting the hair. This technique consists in spraying absorbent particles onto the hair and then in actively brushing the hair in order to remove the particles. However, in general, it is very difficult to obtain complete removal of the particles. The results are not very satisfactory. The hair is not very shiny and it is rough to the touch. One aim of the present invention is in particular to solve such problems.

### DETAILED DESCRIPTION OF THE PREFERRED EMBODIMENTS

Now, subsequent to considerable research carried out on the subject, it has been found by the inventors, entirely unexpectedly and surprisingly, that the above-noted problems of the prior art are addressed by the present invention method for cleansing the hair, which results, at the end of the operation, in hair having a neutral feel and a neutral visual appearance. These and other benefits are obtained by the use of the invention compositions, articles and methods, set forth in more detail below.

In a preferred embodiment, the invention relates to liquid cosmetic compositions, in particular hair compositions, preferably intended for cleansing the hair, comprising:
from 10 to 90% by weight relative to the total weight of the composition of at least one C2-C4 monoalcohol,
from 0.01 to 5% by weight relative to the total weight of the composition of at least one surfactant chosen from anionic surfactants, amphoteric and/or zwitterionic surfactants and nonionic surfactants,
from 0.01 to 5% by weight of an agent for solubilizing liposoluble compounds that is different from the above surfactants and chosen from linear or branched hydrocarbons, hydrogenated or nonhydrogenated polyolefins, volatile silicones, and mixtures thereof,
from 1 to 89.98% of water.
   In these compositions the medium is preferably a cosmetically acceptable aqueous medium.

Hair cleansed with a composition according to the invention has the characteristics of clean hair: it has a natural feel, the hair is shiny (the greasy appearance has disappeared), the hair is airy and light and there is no dandruff. In addition, the hair is more controlled, more flexible and smoother. Furthermore, the treatment removes unpleasant odours.

Thus, a subject of the invention is also the use of the composition as defined above, for cleansing the hair.

A subject of the present invention is also a method for cleansing the hair, said method being characterized in that a composition as defined above is applied to the dry hair, and then in that the hair is dried.

The C2-C4 monoalcohols are linear or branched. They are in particular chosen from ethanol, isopropanol and tert-butanol.

The monoalcohols are preferably present singly or in any mixture of two or more in concentrations ranging from 10 to 50% by weight, and more particularly from 10 to 25% by weight relative to the total weight of the composition.

As nonionic surfactants, use may preferably be made of alkyl polyglycosides, and in particular alkyl polyglucosides (APGs) having an alkyl group comprising from 6 to 30 carbon atoms (C6-C30 alkyl polyglucosides), and preferably 8 to 16 carbon atoms, for instance decyl glucoside (C9/C11 alkyl polyglucoside (1,4)) such as the product sold under the name MYDOL 10 by the company Kao Chemicals, the product sold under the name PLANTAREN 2000 UP or PLANTACARE 2000 UP by the company Henkel, and the product sold under the name ORAMIX NS 10 by the company Seppic; caprylyl/capryl glucoside such as the product sold under the name ORAMIX CG 110 by the company Seppic; lauryl glucoside such as the products sold under the names PLANTAREN 1200 N and PLANTACARE 1200 by the company Henkel; and cocoglucoside such as the product sold under the name PLANTACARE 818/UP by the company Henkel.

As anionic surfactants, use may be made, for example, of carboxylates, amino acid derivatives, alkyl sulphates, alkyl ether sulphates, sulphonates, isethionates, taurates, sulphosuccinates, alkyl sulphoacetates, phosphates and alkylphosphates, polypeptides, anionic derivatives of alkyl polyglucoside, fatty acid soaps, and mixtures thereof.

As carboxylates, mention may be made, for example, of salts (for example alkaline salts) of N-acylamino acids; (C8-C20)alkyl amido ether carboxylates (AECs) such as the sodium lauryl amido ether carboxylate (3 EO) sold under the name AKYPO FOAM 30 by the company Kao Chemicals; (C8-C20)alkyl ether carboxylates, such as the oxyethylenated (6 EO) sodium lauryl ether carboxylate (65/25/10 C12-C14-C16) sold under the name AKYPO SOFT 45 NV by the company Kao Chemicals, oxyethylenated lauryl ether carboxylic acid containing 4.5 EO (AKYPO RLM 45 from KAO); polyoxyethylenated and carboxymethylated olive oil fatty acids, such as the product sold under the name OLIVEM 400 by the company Biologia E Tecnologia; oxyethylenated (6 EO) sodium tridecyl ether carboxylate sold under the name NIKKOL ECTD-6NEX by the company Nikkol; sodium 2-(2-hydroxyalkyloxy)acetate sold under the name BEAULIGHT SHAA by the company Sanyo. Use is preferably made of (C8-C20)alkyl amido ether carboxylic acids and (C8-C20)alkyl ether carboxylic acids and salts thereof.

The amino acid derivatives can be chosen, for example, from sarcosinates and in particular acylsarcosinates, such as sodium lauroylsarcosinate, sold under the name SARKOSYL NL 97 by the company Ciba or sold under the name ORAMIX L 30 by the company Seppic, sodium myristoylsarcosinate, sold under the name NIKKOL SARCOSINATE MN by the company Nikkol, sodium palmitoylsarcosinate, sold under the name NIKKOL SARCOSINATE PN by the company Nikkol; alaninates, such as sodium N-lauroyl-N-methylamidopropionate, sold under the name SODIUM NIKKOL ALANINATE LN 30 by the company Nikkol or sold under the name ALANONE ALE by the company Kawaken and triethanolamine N-lauroyl-N-methylalanine, sold under the name ALANONE ALTA by the company Kawaken; N-acylglutamates, such as triethanolamine monococoylglutamate sold under the name ACYLGLUTAMATE CT-12 by the company Ajinomoto and triethanolamine lauroylglutamate sold under the name ACYLGLUTAMATE LT-12 by the company Ajinomoto; aspartates, such as the mixture of triethanolamine N-lauroylaspartate and of triethanolamine N-myristoylaspartate, sold under the name ASPARACK LM-TS2 by the company Mitsubishi; citrates, and mixtures thereof.

As glycine derivatives, examples include sodium N-cocoylglycinate and potassium N-cocoylglycinate, such as the products sold under the names AMILITE GCS-12 and AMILITE GCK-12 by the company Ajinomoto.

As alkyl ether sulphates, mention may be made, for example, of sodium lauryl ether sulphate (70/30 C12-C14) (2.2 EO), sold under the names SIPON AOS 225 or TEXAPON N702 PATE by the company Henkel, ammonium lauryl ether sulphate (70/30 C12-C14) (3 EO), sold under the name SIPON LEA 370 by the company Henkel, or ammonium (C12-C14)alkyl ether (9 EO) sulphate, sold under the name RHODAPEX AB/20 by the company Rhodia Chimie.

As sulphonates, mention may be made, for example, of alpha-olefin sulphonates, such as sodium alpha-olefin sulphonate (C14-C16), sold under the name BIO-TERGE AS-40 by the company Stepan, sold under the names WITCONATE AOS PROTEGE and SULFRAMINE AOS PH 12 by the company Witco or sold under the name BIO-TERGE AS-40 CG by the company Stepan, secondary sodium olefin sulphonate, sold under the name HOSTAPUR SAS 30 by the company Clariant; or linear alkylaryl sulphonates, such as sodium xylene sulphonate, sold under the names MANROSOL SXS30, MANROSOL SXS40 and MANROSOL SXS93 by the company Manro.

As isethionates, mention may be made, for example, of acylisethionates, such as sodium cocoylisethionate, for instance the product sold under the name JORDAPON CI P by the company Jordan.

As taurates, mention may be made, for example, of the sodium salt of palm kernel oil methyltaurate, sold under the name HOSTAPON CT PATE by the company Clariant; N-acyl-N-methyltaurates, such as sodium N-cocoyl-N-methyltaurate, sold under the name HOSTAPON LT-SF by the company Clariant or sold under the name NIKKOL CMT-30-T by the company Nikkol, sodium palmitoyl methyltaurate, sold under the name NIKKOL PMT by the company Nikkol.

As sulphosuccinates, mention may be made, for example, of oxyethylenated (3 EO) lauryl (70/30 C12-C14) alcohol monosulphosuccinate, sold under the names SETACIN 103 SPECIAL and REWOPOL SB-FA 30 K 4 by the company Witco, the disodium salt of a hemisulphosuccinate of C12-C14 alcohols, sold under the name SETACIN F SPECIAL PASTE by the company Zschimmer Schwarz, oxyethylenated (2 EO) disodium oleamidosulphosuccinate, sold under the name STANDAPOL SH 135 by the company Henkel, oxyethylenated (5 EO) lauramide monosulphosuccinate, sold under the name LEBON A-5000 by the company Sanyo, the disodium salt of oxyethylenated (10 EO) lauryl citrate monosulphosuccinate, sold under the name REWOPOL SB CS 50 by the company Witco, or ricinoleic monoethanolamide monosulphosuccinate, sold under the name REWODERM S 1333 by the company Witco.

As phosphates and alkyl phosphates, mention may be made, for example, of monoalkyl phosphates and dialkyl phosphates, such as lauryl monophosphate, sold under the name MAP 20 by the company Kao Chemicals, the potassium salt of dodecyl phosphate, a mixture of mono- and diester (predominantly diester) sold under the name CRAFOL AP-31 by the company Cognis, the mixture of octyl phosphate monoester and diester, sold under the name CRAFOL AP-20 by the company Cognis, the mixture of ethoxylated (7 mol of EO) 2-butyloctyl phosphate monoester and diester sold under the name ISOFOL 12 7 EO-PHOSPHATE ESTER by the company Condea, the potassium or triethanolamine salt of mono(C12-C13)alkyl phosphate, sold under the references ARLATONE MAP 230K-40 and ARLATONE MAP 230T-60 by the company Uniqema, or potassium lauryl phosphate, sold under the name DERMALCARE MAP XC-99/09 by the company Rhodia Chimie.

The polypeptides are obtained, for example, by condensation of a fatty chain with cereal amino acids and in particular wheat and oat amino acids. As polypeptides, mention may be made, for example, of the potassium salt of hydrolysed lauroyl wheat protein, sold under the name AMINOFOAM W OR by the company Croda, the triethanolamine salt of hydrolysed cocoyl soybean protein, sold under the name MAY-TEIN SY by the company Maybrook, the sodium salt of oat lauroyl amino acids, sold under the name PROTEOL OAT by the company Seppic, collagen hydrolysate grafted onto coconut fatty acid, sold under the name GELIDERM 3000 by the company Deutsche Gelatine, or soybean proteins acylated with hydrogenated coconut acids, sold under the name PROTEOL VS 22 by the company Seppic.

The anionic alkyl polyglucoside derivatives can in particular be citrates, tartrates, sulphosuccinates, carbonates and glycerol ethers obtained from alkyl polyglucosides. Mention may be made, for example, of the sodium salt of cocoylpolyglucoside (1,4) tartaric ester, sold under the name EUCAROL AGE-ET by the company Cesalpinia, the disodium salt of cocoylpolyglucoside (1,4) sulphosuccinic ester, sold under the name ESSAI 512 MP by the company Seppic, the sodium salt of cocoyl-polyglucoside (1,4) citric ester, sold under the name EUCAROL AGE-EC® by the company Cesalpinia. Another anionic holoside derivative may be sodium dodecyl-D-galactoside uronate, sold under the name SODIUM DODECYL-D-GALACTOSIDE URONATE by the company Soliance.

As amphoteric and zwitterionic surfactants, mention may be made, for example, of (C8-C20)alkylbetaines, N—(C8-C20)alkylamidobetaines and their derivatives, glycine derivatives, sultaines, (C8-C20)alkyl polyaminocarboxylates, (C8-C20)alkyl amphoacetates, and mixtures thereof.

As betaines, mention may be made, for example, of cocobetaine, such as the product sold under the name DEHYTON AB-30 by the company Henkel, laurylbetaine, such as the product sold under the name GENAGEN KB by the company Clariant, oxyethylenated (10 EO) laurylbetaine, such as the product sold under the name LAURYLETHER(10 EO)BETAINE by the company Shin Nihon Rica, or oxyethylenated (10 EO)stearylbetaine, such as the product sold under the name STEARYLETHER(10 EO)BETAINE by the company Shin Nihon Rica.

Among N-alkylamidobetaines and their derivatives, mention may be made, for example, of cocamidopropyl betaine, sold under the name LEBON 2000 HG by the company Sanyo, or sold under the name EMPIGEN BB by the company Albright & Wilson, or lauramidopropyl betaine, sold under the name REWOTERIC AMB12P by the company Witco.

As sultaines, mention may be made of cocoylamidopropylhydroxysulphobetaine, sold under the name CROSULTAINE C-50 by the company Croda.

As alkyl polyaminocarboxylates (APACs), mention may be made of sodium cocoylpolyaminocarboxylate, sold under the name AMPHOLAK 7 CX/C and AMPHOLAK 7 CX by the company Akzo Nobel, sodium stearylpolyamidocarboxylate, sold under the name AMPHOLAK 7 TX/C by the company Akzo Nobel, or sodium carboxymethyloleylpolypropylamine, sold under the name AMPHOLAK X07/C by the company Akzo Nobel.

As alkyl amphoacetates, mention may be made, for example, of N-disodium N-cocoyl-N-carboxymethoxyethyl-N-carboxymethylethylenediamine (CTFA name: disodium cocamphodiacetate), such as the product sold under the name MIRANOL C2M CONCENTRE NP by the company Rhodia Chimie, and N-sodium N-cocoyl-N-hydroxyethyl-N-carboxymethylethylenediamine (CTFA name: sodium cocamphoacetate).

The composition may also comprise a mixture of two or more of these surfactants.

The surfactants are preferably chosen from amphoteric and/or zwitterionic surfactants, alkyl polyglycosides and anionic surfactants having at least one carboxylate group.

Preferably, the nature and the concentration of the various surfactants are chosen such that the composition does not foam when it is applied to the hair.

Preferably, the surfactant(s) are present at concentrations ranging from 0.01 to 2%, and more particularly from 0.05 to 1% by weight relative to the total weight of the composition, and even more particularly from 0.075 to 0.5% by weight.

The invention agents for solubilizing liposoluble compounds are not surfactants. These compounds have an HLB of less than 3 (HLB=Hydrophilic-Lipophilic Balance, within the meaning of Griffin; see J. Soc. Cosm. Chem. 1954 (Vol. 5), pp 249-256; balance between the hydrophilic nature and the lipophilic nature).

The agents for solubilizing liposoluble compounds are preferably volatile.

For the purpose of the present invention, the term “volatile” is intended to mean any compound having a boiling point below 290° C. at atmospheric pressure.

Examples of invention agents for solubilizing liposoluble compounds follow, and include hydrocarbons, polyolefins, and volatile silicones.

The hydrocarbons preferably have from 8 to 16 carbon atoms, and preferably branched. They are more particularly C10-C14 isoparaffins. The polyolefins are in particular poly-α-olefins, and more particularly:
of hydrogenated or nonhydrogenated polybutene type, and preferably hydrogenated or nonhydrogenated polyisobutene.

Use is preferably made of isobutylene oligomers of molecular weight less than 1000.

By way of examples of poly-α-olefins that can be used in the context of the present invention, mention may more particularly be made of the polyisobutenes sold under the name PERMETHYL 99 A, 101 A, 102 A, 104 A (n=16) and 106 A (n=38) by the company Presperse Inc, or alternatively the product sold under the name ARLAMOL HD (n=3) by the company ICI (n denoting the degree of polymerization),
of hydrogenated or nonhydrogenated polydecene type.

Such products are sold, for example, under the names ETHYLFLO by the company Ethyl Corp., and ARLAMOL PAO by the company ICI.

The volatile silicones are more particularly chosen from those having a boiling point of between 60° C. and 260° C., and even more particularly from:

(i) cyclic silicones containing from 3 to 7 silicon atoms, and preferably 4 to 5. They are, for example, octamethylcyclotetrasiloxane, sold in particular under the name “VOLATILE SILICONE 7207” by Union Carbide or “SILBIONE 70045 V 2” by Rhodia Chimie, or decamethylcyclopentasiloxane, sold under the name “VOLATILE SILICONE 7158” by Union Carbide or “SILBIONE 70045 V 5” by Rhodia Chimie, and mixtures thereof.

(ii) linear volatile silicones containing 2 to 9 silicon atoms and having a viscosity of less than or equal to 5×10-6 m2/s at 25° C. This is, for example, decamethyltetrasiloxane, sold in particular under the name “SH 200” by the company Toray Silicone. Silicones which fall into this class are also described in the article published in Cosmetics and Toiletries, Vol. 91, January 76, 27-32—Todd & Byers “Volatile Silicone Fluids for Cosmetics”.

The agent(s) for solubilizing liposoluble compounds are preferably present at concentrations ranging from 0.1 to 3% by weight, and more particularly from 0.5 to 1.5% by weight relative to the total weight of the composition.

The composition may also comprise adjuvants such as those conventionally used in the fields under consideration, such as, for example, organic solvents, hydrophilic or lipophilic thickeners and gelling agents, softeners, antioxidants, opacifiers, stabilizing agents, chelating agents, fragrances, screening agents, essential oils, dyestuffs, pigments, vitamins, provitamins, anti-dandruff agents, antiseborrheic active agents which allow excess sebum on the hair to be cleansed, and antimicrobial agents which remove from the hair the microorganisms which are possibly present thereon, anti-hairloss agents, active agents that promote hair growth, hydrophilic or lipophilic active agents, lipid vesicles optionally encapsulating one or more active agents, or any other ingredient normally used in cosmetics or dermatology. It may optionally also contain preserving agents. The amounts of the various constituents of the composition according to the invention are typically those conventionally used in the fields under consideration. Of course, these adjuvants should be such that, and should be used in amounts such that, they do not disturb the cleansing of the hair according to the invention. The amount of these adjuvants can range, for example, from 0.01 to 30% by weight relative to the total weight of the composition.

The composition may also contain an additive chosen from preserving agents, pH regulators and fragrances, for example.

The viscosity of the cosmetic composition according to the invention is preferably less than 1500 mPa·s, and more preferably less than 1000 mPa·s. The viscosity is measured at 25° C. with a Rheomat RM 180 device.

The pH of the compositions used according to the invention is generally between 2 and 12, preferably from 3 to 8.

The compositions according to the invention preferably comprise from 40 to 89% by weight of water, more particularly from 50 to 85% by weight, and even more preferentially from 60 to 85% by weight, and preferentially from 70 to 85% by weight, relative to the total weight of the composition.

The compositions according to the invention can be prepared according to techniques well known to those skilled in the art in the field.

The composition can be applied in any fashion/manner, for example with the hands, with the tip of an applicator, with a spraying device, with a dispensing comb, and preferably with an insoluble substrate impregnated with the composition.

The inventors have found that the use of an insoluble substrate for applying the composition gives particularly advantageous results.

Thus, according to a preferred embodiment of the invention, the compositions according to the invention can also be used for impregnating water-insoluble substrates so as to constitute articles, and in particular towelettes or gloves intended for cleansing the hair. The water-insoluble substrate may comprise one or more layers and it may be chosen from the group comprising woven materials, nonwoven materials, foams, sponges or cotton wool, as sheets, balls or films. It may in particular be a nonwoven substrate based on fibres of natural origin (flax, wool, cotton, silk) or of synthetic origin (cellulose derivatives, viscose, polyvinyl derivatives, polyesters such as polyethylene terephthalate, polyolefins such as polyethylene or polypropylene, polyamides such as Nylon, acrylic derivatives). The nonwovens are described in general in Riedel “Nonwoving Bonding Methods & Materials”, Nonwoven World (1987). These substrates are obtained according to the usual processes of the technique for preparing nonwovens.

When the substrate is a nonwoven, use is preferably made of a thick nonwoven, which does not roll up into a ball and which is solid enough not to disintegrate and not to pill on application to the skin. It should be absorbent, soft, at least on one face, in particular for removing makeup from the eyes. As suitable nonwovens, mention may be made, for example, of those sold under the names Ultraloft 15285-01, Ultraloft 182-008, Ultraloft 182-010 and Ultraloft 182-016 by the company BBA, and Vilmed M1519 Blau, Vilmed M 1550 N and 112-132-3 by the company Freudenberg, that sold under the name Norafin 11601-010B by the company Jacob Holm Industries, that sold under the name Aquadim VE50 G1NL (spun lace) by the company Tharreau, and the flocked nonwovens sold under the names Univel 109 and Univel 119 by the company Uni Flockage.

Moreover, this substrate may comprise one or more layers having identical or different properties and may have properties of elasticity and of softness and other properties suitable for the desired use. The substrates may comprise, for example, two components having different elasticity properties, as described in document WO-A-99/13861, or may comprise a single layer with different densities, as described in document WO-A-99/25318, or may comprise two layers of different textures, as described in document WO-A-98/18441.

This article preferably constitutes a glove, but may also be in another form (towelette, for example).

A subject of the invention is therefore also an article comprising a water-insoluble substrate and a composition as defined above, impregnated with said substrate.

A subject of the invention is also the use of the composition as defined above, for preparing an article intended for cleansing the hair.

According to another aspect of the present invention, a single-use cosmetic article is prepared, comprising at least one substrate impregnated with a liquid cosmetic composition as defined above, said substrate being permeable and comprising at least a first group of fibres and at least a second group of fibres, the hydrophilicity of which is different from that of the fibres of the first group, said first and second groups of fibres being arranged such that a first face of the substrate exhibits a hydrophilicity greater than the hydrophilicity of a second face of the substrate, that is opposite the first, said first face being external to the substrate so as to be able to be brought into contact with a surface to be treated, in particular the hair.

With such an article, the gain in effectiveness of a cosmetic treatment can be substantially improved by allowing a greater amount of the composition with which the article is impregnated to be released on the surface to be treated.

The term “permeable” denotes a substrate through whose entire thickness said cosmetic composition can pass. Thus, unlike some articles of the prior art to which reference was made above, the article according to the invention does not comprise an impermeable layer which may form a barrier to the composition with which it is impregnated.

Of course, the substrate is water-insoluble, i.e. its integrity is not substantially affected by the presence of the aqueous cosmetic composition with which it is impregnated, during at least the entire lifetime of the product.

Advantageously, the substrate is a monolayer substrate. The term “monolayer substrate” denotes a substrate where all the fibres of which it is composed are linked to one another during the same linking process. At the end, in the thickness of the substrate, the distribution of the two types of fibres is non-homogeneous and, according to one profile, may be characterized by:
a majority of fibres of the first group on the first face of the substrate;
a majority of fibres of the second group on the second face of the substrate, and, between the two faces,
a relative amount of fibres of the first group that varies according to a relatively progressive profile, the slope of which is of opposite sign to that of the relatively progressive profile corresponding to the relative amount of fibres of the second group.

Such a structure differs from multilayer structures in which various layers are formed and bound separately, and then bonded to one another by thermal bonding, hot-welding or by ultrasound.

Advantageously, the first face of the substrate has a release index under static pressure (RISP1), the second face of the substrate having a release index under static pressure (RISP2), the RISP1/RISP2 ratio being greater than or equal to 1.5.

Release Index Under Static Pressure (RISP):

The test that will be described hereinbelow makes it possible to measure the release index under static pressure (RISP) of the two faces of an article impregnated with a cosmetic product as defined above. The RISP determines the amount of liquid impregnated in the support that may be released by each of the two faces of the support under the effect of a static pressure, and thereby reveals whether there is a difference in release between the 2 faces.

Since the gesture usually adopted by the consumer during the use of the article consists in wiping his or her skin or hair with a first face of the article, the other face of said article being in contact with the hand, it is desirable to determine the amount of liquid simultaneously released by the two faces of the support.

To this end, two sheets of absorbent material of paper towel type, of brand name Wypall L30 (reference 7303) sold by Kimberley-Clark, with a basis weight of 50 g/m2, are accurately weighed to the nearest hundredth of a gram, using a Mettler Toledo PR5002 balance. The sheets of absorbent material must be at least as large as the article to be tested, and preferably slightly larger such that they overlap the outer edge of the support by at least two centimetres.

The first sheet is placed on a glass plate having dimensions at least as large as those of the article to be tested. The article to be tested is placed on the first sheet of Wypall L30. The second sheet of Wypall L30 is placed on top.

A metal plate with a surface area at least equal to the surface area of the article to be tested, and weighing 3.7 kg, is placed on the assembly. After one minute, the plate is removed and each sheet of Wypall L30 is weighed.

The weight of liquid that has impregnated each sheet of Wypall L30 is calculated by means of the difference in mass of the Wypall L30 after and before the test. This weight of liquid corresponds to the amount of liquid that has been released by each of the two faces of the support, which are noted, respectively, as RISP1 for the face having the higher hydrophilicity and RISP2 for the face having the lower hydrophilicity.

The ratio between RISP1 and RISP2 indicates the difference in release between the 2 faces of the towelette.

The value retained for the RISP1/RISP2 ratio is the mean value of measurements determined on four articles.

Advantageously, the RISP1/RISP2 ratio ranges from 1.5 to 15, preferably from 2.5 to 10, and more preferably from 2.5 to 7.

Obviously, when the article is hollow and is configured especially in the form of a glove, the two sheets of which it is composed are separated and the test is performed on the sheets thus separated.

Substrate:

The substrate is preferably manufactured according to the water-jet bonding process conventionally used for the preparation of nonwovens.

The first step is the production of webs of fibres having different hydrophilicities. These webs may be prepared according to various methods known to those skilled in the art, for example by carding using balls of fibres, or by extrusion of polymer in the form of continuous filaments of fibres, or via an aero-pneumatic route.

The web of fibres that are the most hydrophilic and the web of fibres that are the least hydrophilic are placed on a conveyor belt and conveyed under arrays of jets of water at high pressure, which bind the fibres together. These high-pressure water jets cause rearrangement of the fibres in three directions within the fibrous structure, thus leading to overlapping of the fibres, binding them together without any addition of chemical binding agents. Generally, the webs of fibres pass under several arrays of water jets whose pressure gradually increases from the first array to the last. This technique is known as hydrobonding.

The substrate may, moreover, be embossed, hemstitched, calendered or printed on, or may undergo any type of line-end treatment. Thus, advantageously, one of said faces of the support, in particular the least hydrophilic face, is rendered exfoliant, in particular by hot-calendering.

According to this particular process, it should be noted that, although formed from two webs of fibres, in the end, the substrate comprises only one layer. The risks of delamination that may have been observed in multilayer structures to which reference was made above are eliminated.

The most hydrophilic fibres may be chosen from cotton, cellulose or viscose fibres.

The least hydrophilic fibres may be chosen from polypropylene, polyester, polyamide or polyethylene fibres.

According to another aspect of the present invention, a single-use cosmetic article is prepared, comprising at least one substrate impregnated with a liquid cosmetic composition, said substrate being permeable and comprising at least a first group of fibres and at least a second group of fibres, the hydrophilicity of which is different from that of the fibres of the first group, said first and second groups of fibres being arranged such that a first face of the substrate, external to the latter, exhibits a hydrophilicity greater than the hydrophilicity of a second face of the substrate, opposite the first, said article comprising at least one hollow or concave portion, said hollow or concave portion being intended:
i) either to receive a surface to be treated so as to bring said surface into contact with an inner surface of the article;
ii) or to receive all or part of a hand, so as to bring an outer surface of the article into engagement with a surface to be treated.

Such a hollow or concave article may be configured in the form of a glove, a mitten, a slipper, a fingerstall, a bonnet, a skullcap or a headcap.

Such a hollow article may be obtained by assembling at least “two sheets” along their respective edges, or by folding a sheet on itself and then fixing on themselves the edges of the sheet thus folded.

When the hollow or concave portion is intended to receive all or part of a hand, in order to bring an outer surface of the article into engagement with a surface to be treated, the most hydrophilic face is on the outside of the hollow or concave portion.

Advantageously, the substrate is impregnated with said cosmetic composition according to a degree ranging from 100% to 1000%, preferably from 150% to 800%, and more preferably from 150% to 400% by weight of composition relative to the weight of non-impregnated substrate.

The following examples will make it possible to understand the invention more thoroughly, without however being limiting in nature. The amounts indicated are in % by weight, unless otherwise mentioned.

In the examples which follow, given by way of nonlimiting illustration, concrete compositions in accordance with the invention are given (AM means active material).

### EXAMPLE 1

### Hair-Cleansing Glove

The cleansing composition described below is impregnated to 200% onto a Sandler nonwoven ref. Mar. 2, 1001.

| Ingredients: | % by mass: |
|---|---|
| Water | qs 100% |
| Ethanol | 16% AM |
| Disodium cocoamphodiacetate | 0.2% AM |
| (MIRANOL C2M CONC from RHODIA CHIMIE) | |
| C11-C12 isoparaffin (ISOPAR H from EXXON | 1% |
| MOBIL CHEMICAL) | |
| Fragrance, preserving agent | qs |
| pH agent qs | pH 7 |

The Sandler nonwoven ref. Mar. 2, 1001 is obtained by hydrobonding and is composed of 100% of viscose fibres on one face and of 100% of polypropylene (PP) fibres on the other face. It is cut into the shape of a mitten or a glove, the PP faces of which are welded internally via the edges so as to delimit a pocket into which to introduce the hand.

On hair that was washed a few days ago, half a head is massaged with a glove comprising the composition according to the invention. The hair is massaged for approximately 30 seconds and then the hair is allowed to dry in the open air.

After drying, on the left (invention), the hair is cleaner and more airy than on the right without treatment. It also has a more pleasant feel on the left (invention) than before treatment.

The hair is smoother to the touch, lighter and more flexible. It has a greater volume and is easier to style.

### EXAMPLE 2

### Hair-Cleansing Glove

The cleansing composition described below is impregnated to 200% onto a Sandler nonwoven ref. Mar. 2, 1001.

| Ingredients: | % by mass: |
|---|---|
| Water | qs 100 |
| Denatured ethanol (93.1% AM in water) | 20% |
| Disodium cocoamphodiacetate | 0.2% AM |
| (MIRANOL C2M CONC from RHODIA CHIMIE) | |
| Hydrogenated polyisobutene | 5% |
| Fragrance | qs |
| pH agent qs | pH 7 |

The Sandler nonwoven ref. Mar. 2, 1001 is obtained by hydrobonding and is composed of 100% of viscose fibres on one face and of 100% of polypropylene (PP) fibres on the other face. It is cut into the shape of a mitten or a glove, the PP faces of which are welded internally via the edges so as to delimit a pocket into which to introduce the hand.

### EXAMPLE 3

### Hair-Cleansing Glove

The cleansing composition described below is impregnated to 200% onto a Sandler nonwoven ref. Mar. 2, 1001.

| Ingredients: | % by mass: |
|---|---|
| Water | qs 100 |
| Ethanol | 16% |
| Lauryl ether carboxylic acid | 0.4% AM |
| containing 4.5 mol of ethylene oxide | |
| (AKYPO RLM 45 from Kao) | |
| C11-C12 isoparaffin (ISOPAR H from EXXON | 1% |
| MOBIL CHEMICAL) | |
| Fragrance, preserving agent | qs |
| pH agent qs | pH 7 |

The hair is softer to the touch, lighter and more flexible. It has more volume and is easier to style.

The above written description of the invention provides a manner and process of making and using it such that any person skilled in this art is enabled to make and use the same, this enablement being provided in particular for the subject matter of the appended claims, which make up a part of the original description and including a cosmetic hair composition comprising, in a cosmetically acceptable aqueous medium:
from 10 to 90% by weight relative to the total weight of the composition of at least one C2-C4 monoalcohol,
from 0.01 to 5% by weight relative to the total weight of the composition of at least one surfactant chosen from anionic surfactants, amphoteric and/or zwitterionic surfactants and nonionic surfactants,
from 0.01 to 5% by weight of an agent for solubilizing liposoluble compounds, said agent being chosen from linear or branched hydrocarbons, hydrogenated or nonhydrogenated polyolefins, volatile silicones, and mixtures thereof,
from 1 to 89.98% of water.

Preferred embodiments of the invention similarly fully described and enabled include a single-use cosmetic article comprising at least one substrate impregnated with an invention liquid cosmetic composition, said substrate being permeable and comprising at least a first group of fibres and at least a second group of fibres, the hydrophilicity of which is different from that of the fibres of the first group, said first and second groups of fibres being arranged such that a first face of the substrate exhibits a hydrophilicity greater than the hydrophilicity of a second face of the substrate, opposite the first, said first face being external to the substrate so as to be able to be brought into contact with a surface to be treated, in particular the hair.

As used herein, the phrases “selected from the group consisting of,” “chosen from,” “at least one selected from the group consisting of” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising:
from 10 to 90% by weight relative to the total weight of the composition of at least one C2-C4 monoalcohol,
from 0.01 to 5% by weight relative to the total weight of the composition of at least one surfactant selected from the group consisting of anionic surfactants, amphoteric and/or zwitterionic surfactants and nonionic surfactants,
from 0.01 to 5% by weight of at least one agent for solubilizing liposoluble compounds selected from the group consisting of linear or branched hydrocarbons, hydrogenated or nonhydrogenated polyolefins, and volatile silicones,
from 1 to 89.98% of water.

2. The composition according to claim 1, comprising at least one C2-C4 monoalcohol selected from the group consisting of ethanol, isopropanol and tert-butanol.

3. The composition according to claim 1, wherein the monoalcohol(s) is(are) present in concentrations ranging from 10 to 50% by weight relative to the total weight of the composition.

4. The composition according to claim 1, comprising at least one surfactant selected from the group consisting of amphoteric and/or zwitterionic surfactants, alkyl polyglucosides and anionic surfactants having at least one carboxylate group.

5. The composition according to claim 1, comprising at least one amphoteric and/or zwitterionic surfactant are selected from the group consisting of alkyl amphoacetates.

6. The composition according to claim 1, comprising at least one anionic surfactant selected from the group consisting of (C8-C20)alkyl amido ether carboxylic acids, salts thereof, (C8-C20)alkyl ether carboxylic acids, and salts thereof.

7. The composition according to claim 1, wherein the surfactant(s) are present at concentrations ranging from 0.01 to 2% by weight relative to the total weight of the composition.

8. The composition according to claim 1, wherein the solubilizing agent(s) are present at concentrations ranging from 0.1 to 3% by weight relative to the total weight of the composition.

9. A method, comprising applying the composition of claim 1 to human hair.

10. The method of claim 9, wherein the hair is wet when said composition is applied thereto.

11. The method of claim 9, wherein the hair is dry when said composition is applied thereto.

12. An article, comprising at least one substrate impregnated with the composition of claim 1.

13. A article comprising at least one substrate impregnated with a composition comprising:
from 10 to 90% by weight relative to the total weight of the composition of at least one C2-C4 monoalcohol,
from 0.01 to 5% by weight relative to the total weight of the composition of at least one surfactant selected from the group consisting of anionic surfactants, amphoteric and/or zwitterionic surfactants and nonionic surfactants,
from 0.01 to 5% by weight of at least one agent for solubilizing liposoluble compounds selected from the group consisting of linear or branched hydrocarbons, hydrogenated or nonhydrogenated polyolefins, and volatile silicones,
from 1 to 89.98% of water,
said substrate being permeable and comprising at least a first group of fibres and at least a second group of fibres, the hydrophilicity of said second group of fibres being different from that of the fibres of the first group, said first and second groups of fibres being arranged such that a first face of the substrate exhibits a hydrophilicity greater than the hydrophilicity of a second face of the substrate, opposite the first, said first face being external to the substrate so as to be able to be brought into contact with a surface to be treated.

14. The article according to claim 12, wherein the substrate is a monolayer substrate.

15. The article according to claim 12, wherein the substrate is impregnated with said composition according to a degree from 100% to 1000% by weight of composition relative to the weight of non-impregnated substrate.

16. The article according to claim 13, wherein the substrate is impregnated with said composition according to a degree from 100% to 1000% by weight of composition relative to the weight of non-impregnated substrate.

17. The article according to claim 13, wherein the substrate is a monolayer substrate.

18. The article according to claim 13, wherein the first face of the substrate has a release index under static pressure (RISP1), the second face of the substrate has a release index under static pressure (RISP2), and the RISP1/RISP2 ratio is from 1.5 to 15

19. A method, comprising contacting dry hair with the article of claim 12.

20. A method, comprising contacting dry hair with the article of claim 13.
